(19) United States
(12) Patent Application Publication
(10) Pub. No.: US 2018/0220997 A1
(43) Pub. Date: Aug. 9, 2018

(54) SYSTEM AND METHOD FOR ACCELERATED CLUTTER FILTERING IN ULTRASOUND BLOOD FLOW IMAGING USING RANDOMIZED ULTRASOUND DATA

(71) Applicant: MAYO FOUNDATION FOR MEDICAL EDUCATION AND RESEARCH, Rochester, MN (US)

(72) Inventors: Pengfei Song, Rochster, MN (US); Joshua D. Trzasko, Rochester, MN (US); Armando Manduca, Rochester, MN (US); Shigao Chen, Rochester, MN (US)

(21) Appl. No.: 15/887,029

(22) Filed: Feb. 2, 2018

Related U.S. Application Data
(60) Provisional application No. 62/454,213, filed on Feb. 3, 2017.

(51) Int. Cl. A61B 8/08; A61B 8/06

(52) U.S. Cl. CPC A61B 8/5269; A61B 8/488; A61B 8/06

(57) ABSTRACT

Described here are systems and methods for ultrasound clutter filtering to produce images of blood flow in a subject. In general, the clutter filtering is based on a singular value implementation, such as an accelerated singular value decomposition (“SVD”). In one example, the singular value-based clutter filtering can be accelerated by implementing a randomized SVD (“rSVD”). In another example, the singular value-based clutter filtering can be accelerated by implementing a randomized spatial downsampling. In still another example, singular value-based clutter filtering can be accelerated by implementing both an rSVD and a randomized spatial downsampling.

### CROSS-REFERENCE TO RELATED APPLICATIONS

This application claims the benefit of U.S. Provisional Patent Application Ser. No. 62/454,213, filed on Feb. 3, 2017, and entitled “SYSTEM AND METHOD FOR ACCELERATED CLUTTER FILTERING IN ULTRASOUND BLOOD FLOW IMAGING USING RANDOMIZED ULTRASOUND DATA,” which is herein incorporated by reference in its entirety.

### BACKGROUND

The emergence of software beamforming-based ultrasound systems has allowed for advancements in the field of ultrasound blood flow imaging. A typical software beamforming, high frame-rate ultrasound system can collect tens of thousands of frames of ultrasound data within a second, which is hundreds of times more than conventional ultrasound systems, which has a frame rate or pulse repetition frequency (“PRF”) on the order of 20-100 Hz). The sheer amount of high frame-rate ultrasound data provides extremely rich spatiotemporal information of the interrogated tissue, which can be used to finely differentiate blood signals from tissue clutter signals (i.e., clutter filtering) for high resolution microvessel imaging.

However, the large amount of high frame-rate ultrasound data being generated (on the scale of hundreds of megabytes to several gigabytes per second) also places high demands on the clutter filter processing, especially for singular value-based clutter filter techniques. At present, this high computational demand remains as a significant hurdle for real-time implementation of singular value-based clutter filters for high frame-rate blood flow imaging.

### SUMMARY OF THE DISCLOSURE

The present disclosure addresses the aforementioned drawbacks by providing a method for producing an image of blood flow using an ultrasound imaging system. The method includes providing ultrasound data acquired from a subject with the ultrasound imaging system and forming randomized data by randomizing the ultrasound data. In one example, the randomized data can be formed by multiplying the data by a random matrix. In another example, the randomized data can be formed based on a random downsampling of the ultrasound data. Blood flow signal data are estimated from the ultrasound data by clutter filtering tissue signals from the ultrasound data using the randomized data. An image of blood flow in the subject is then produced from the estimated blood flow signal data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for producing an image depicting blood flow in a subject using an ultrasound system.

FIG. 2 depicts ultrasound data frames and the corresponding data ensembles that can be acquired with a high frame rate ultrasound system.

FIG. 3 is a flowchart setting forth the steps of an example method for accelerated clutter filtering of ultrasound data using a randomized SVD of the ultrasound data.

FIG. 4 depicts an example plot illustrating the effect of power iteration on the approximation accuracy of randomized singular value decomposition-based clutter filtering.

FIG. 5 is a flowchart setting forth the steps of an example method for accelerated clutter filtering of ultrasound data using a randomized spatial downsampling of the ultrasound data.

FIG. 6 depicts an example of randomly downsampling ultrasound data based on different random downsampling patterns.

FIG. 7 depicts an example of a random downsampling pattern that is based on a Poisson distribution.

FIG. 8 depicts an example blood flow images generated using clutter filtering based on randomized spatial downsampling of the ultrasound data.

FIG. 9 depicts an example of downsampling ultrasound data based on different structured downsampling patterns.

FIG. 10 is a block diagram of an example ultrasound imaging system that can implement the methods described here.

### DETAILED DESCRIPTION

Described here are systems and methods for ultrasound clutter filtering to produce images of blood flow in a subject. The systems and methods described in the present disclosure may be advantageously applied to fast ultrasound imaging techniques, including ultrafast plane wave imaging techniques. In general, the clutter filtering is based on a singular value implementation, such as an accelerated singular value decomposition (“SVD”). In one example, the singular value-based clutter filtering can be accelerated by implementing a randomized SVD (“rSVD”). In another example, the singular value-based clutter filtering can be accelerated by implementing a randomized spatial downsampling. In still another example, singular value-based clutter filtering can be accelerated by implementing both an rSVD and a randomized spatial downsampling.

The computational complexity of a full SVD of a rank-k matrix is roughly O(mnk), where m and n are the first and second dimensions of the rank-k matrix. The rSVD methods described here approximate the full SVD by capturing the first k singular values of the ultrasound data matrix, with a computational complexity of O(mn log(k)+(m+n)²). The randomized spatial downsampling methods described here take advantage of the redundancy of high frame-rate ultrasound data. In general the randomized spatial downsampling methods described here include downsampling the ultrasound data matrix. The ultrasound data matrix may generally have at least two spatial dimensions (e.g., x and y) and one temporal dimension (e.g., t), and may be reshaped to form a Casorati matrix with a dimension m×n, where m=xy and n=t. Thus, as one example, an ultrasound data matrix that has been reshaped as a Casorati matrix can be downsampled from an m×n matrix to an (m/p)×n matrix, where p is a positive integer. Using the randomized spatial downsampling, the complexity of the SVD used for clutter filtering is reduced to O(mnk/p) without significant alterations to the distribution of singular values of each downsampled matrix (i.e., the downsampled matrices are still with rank-k). By parallel processing the downsampled matrices, an acceleration factor of p can be reached.

Thus, the systems and methods described here are capable of improving the operation of ultrasound imaging systems used for blood flow imaging. As one example, using the methods described in the present disclosure, the computational burden of clutter filtering can be reduced, thereby allowing the use of robust clutter filtering techniques to a wider range of ultrasound imaging system hardware. As another example, the reduced computational burden of the methods described in the present disclosure allow for faster processing of ultrasound data, which enables real-time clutter filtering and reconstruction of blood flow images with high frame rates.

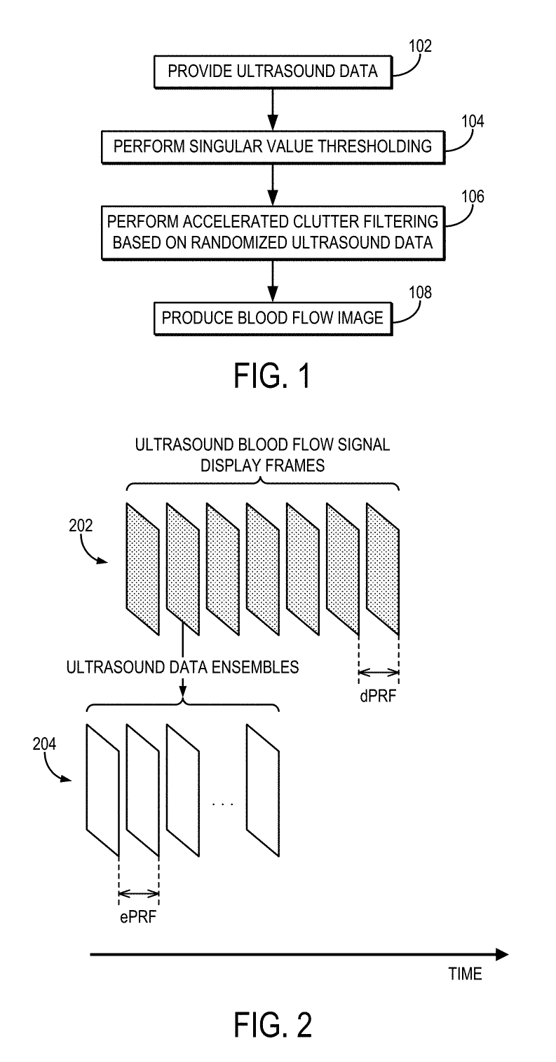

Referring now to FIG. 1, a flowchart is illustrated as setting forth the steps of an example method for producing an image depicting blood flow in a subject using an ultrasound system. The method includes providing ultrasound data to a computer system for processing, as indicated at step 102. Providing the ultrasound data to the computer system may include acquiring ultrasound data with an ultrasound system, or may include retrieving previously acquired ultrasound data from a data storage or memory. The computer system may be a part of the ultrasound system, or may be a separate computer system. The acquired ultrasound data can have any suitable form, and in one example may be ultrasound radio frequency (“RF”) signal data. In another example, the ultrasound data may be in-phase quadrature (“IQ”) signal data. The ultrasound data can also be of other forms of ultrasound data, such as ultrasound data that include information from a pulse-echo ultrasound data acquisition.

As one example, ultrasound data designated for blood flow imaging can be continuously acquired by the ultrasound system for real-time display, as shown in FIG. 2. In this example, the ultrasound blood flow signal display frames 202 are refreshed at a certain frame rate or pulse-repetition-frequency (“PRF”) for continuous monitoring of the blood flow signal. Each ultrasound blood flow signal display frame is derived from multiple data ensembles 204 that are being collected at a higher frame rate or PRF (e.g., several hundreds to tens of thousands of ensembles per second) for blood flow signal processing. The ensemble PRF (or frame rate) can be referred to as “ePRF” and the display PRF (or frame rate) can be referred to as “dPRF.”

The number of ensembles per ultrasound blood flow signal display frame depends on the desired dPRF of ultrasound blood flow imaging. Different ultrasound blood flow signal display frames can have mutually exclusive data ensembles, or can include the same ultrasound data ensembles (e.g., certain data ensembles are assigned to different consecutive frames in an overlapped sliding-window fashion) to fulfill a certain dPRF requirement. In general, the dPRF should be smaller or equal to ePRF.

Before clutter filtering, the ultrasound data can be processed using an adaptive singular value thresholding (“SVT”) process, as indicated at step 104, to determine an adaptive clutter filter cutoff value, such as by using the methods described in co-pending International Patent Application Serial No. PCT/US2017/16190, which is herein incorporate by reference in its entirety. The SVT can also be manually selected or otherwise determined by a user, as described below.

After an SVT value is determined, the ultrasound data are processed using accelerated clutter filtering, as indicated at step 106. In general, the accelerated clutter filtering implements a randomization of the ultrasound data. As mentioned above and described below in more detail, in some instances the accelerated clutter filtering implements randomized ultrasound data using an rSVD of the ultrasound data, and in some other instances the accelerated clutter filtering implements randomized ultrasound data using a randomized spatial downsampling of the ultrasound data. As a result of the processing performed in steps 104 and 106, blood flow signal data are estimated, from which an image of the blood flow in the subject can be reconstructed or otherwise produced, as indicated at step 108.

The blood flow signal data can also be processed to suppress the tissue signal and noise, register the blood flow signals from different ultrasound data frames to suppress the physiologic or operator-induced motion, and to calculate the desired blood flow signals such as color Doppler, power Doppler, spectral Doppler, vector Doppler, and so on. To reveal fine details of the microvessel and to obtain a high resolution microvessel image, a long acquisition of ultrasound ensembles is usually necessary to accumulate sufficient microvessel blood flow signal. However, in real-time applications, such accumulation will result in a decreased blood flow imaging frame rate (i.e., decreased dPRF). An alternative approach to accumulation is to accumulate blood flow signal from each ultrasound data frame 202 either during the real-time display (e.g., the concurrent display is obtained by accumulating the blood flow signal from the last 10 frames) or retrospectively with the real-time display and data acquisition halted, such as by retrospectively accumulating the previous 100 frames of blood flow signal to obtain a high resolution blood flow image. Before accumulation, a registration can be performed to remove the physiologic and operator-induced motion so that the final high resolution blood flow image will not be blurred or corrupted. As one non-limiting example, such registration can be performed based on the brightness image (i.e., B-mode) of the blood flow image from each frame.

The singular value thresholding (“SVT”) performed in step 104 can include performing adaptive SVT. As one specific, and non-limiting, example, the adaptive SVT cutoff selection can implement one or more of the methods described in co-pending International Patent Application Serial No. PCT/US2017/16190, which is herein incorporated by reference in its entirety. In such a process, a determination is first made whether an external SVT cutoff value is being used, such as a cutoff adjustment input from the ultrasound system control panel or interface. If such an external cutoff value is being used, then that cutoff value is provided to the computer system from an external input by the user.

If an external cutoff value is not to be used, then the computer system determines the SVT cutoff adaptively and automatically. As one example, the computer system can check an adjustment flag. If the flag is true, the computer system will calculate a new adaptive SVT cutoff value and update the existing cutoff value. If the flag is false, the computer system will use the currently computed SVT cutoff value. The adjustment flag can include at least one of a certain time interval (e.g., updating the SVT cutoff value once every second), a certain number of display frames or data ensembles (e.g., updating the SVT cutoff value once every 10 frames have been displayed), an external input command (e.g., external user request for updating the SVT cutoff value), and so on.

Having described a general process for producing an image that depicts blood flow in a subject based on clutter filtered ultrasound data, methods for implementing accelerated clutter filtering based on randomized ultrasound data are now described. As one example, accelerated clutter filtering can implement a randomized SVD (“rSVD”) of the ultrasound data. In another example, the accelerated clutter filtering can implement a randomized spatial downsampling of the ultrasound data. In still another example, the accelerated clutter filtering can implement both an rSVD and a randomized spatial downsampling of the ultrasound data.

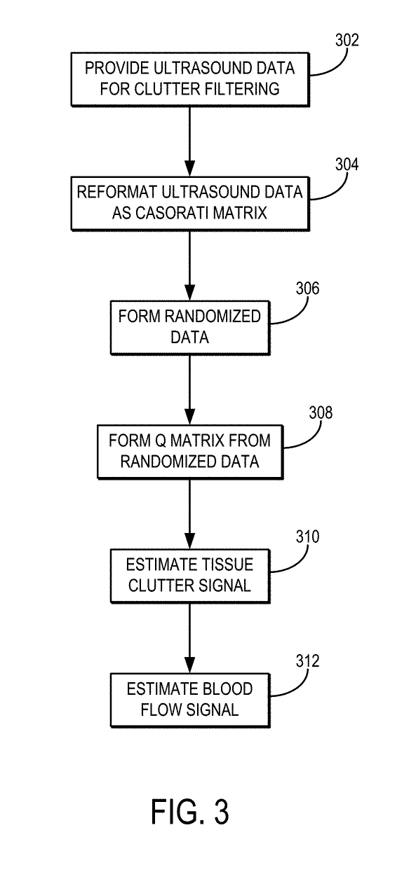

Referring now to FIG. 3, a flowchart is illustrated as setting forth the steps of an example method for accelerated clutter filtering of ultrasound data using an rSVD of the ultrasound data. The original ultrasound data matrix, S, which has dimension x×y×t (where X and y correspond to lateral and axial dimensions, respectively, and t corresponds to the temporal dimension) corresponding to the data acquired with the ultrasound system are provided for processing, as indicated at step 302. The ultrasound data matrix is reformatted as a Casorati matrix with dimension xy×t before processing, as indicated at step 304. As described above, the dimensions of the Casorati matrix can also be defined as m×n with m=xy and n=t. In instances where the ultrasound data include three spatial dimensions (i.e., x, y, and z), the Casorati matrix will have dimensions m m×n with m=xyz and n=t.

The rSVD method described here calculates a desired rank-k approximation (i.e., the subspace in which the tissue clutter is assumed or otherwise expected to lie) of the ultrasound data matrix, S, by forming a matrix, [symbol] , with dimension m×k whose columns form an approximate orthonormal basis for the column space of S. To form this [symbol] matrix, the ultrasound data matrix is first multiplied by a random matrix, Ω, as indicated at step 306 to form the following randomized data matrix:

S′=sΩ (1).

The randomized data, S′, can serve as an approximate basis for the column space of the original ultrasound data, S. As one example, the entries in the random matrix follow a standard normal distribution, N(0,1). The random matrix, Ω, has dimension n×(k+r), where r is the extra rank to be calculated to improve the approximation accuracy of the rSVD implementation. In general, r≥0, and in some examples may be equal to 1 or 2.

The [symbol] matrix can then be formed, as indicated at step 308, where again the columns of the [symbol] matrix form an approximate orthonormal basis for the column space of the original ultrasound data, S. When forming the [symbol] matrix, steps including increasing the decay rate of the singular value curve and orthonormalization can be added to improve the accuracy of the rSVD. The [symbol] matrix can be formed by QR-factorization (also known as QR-decomposition), SVD, and so on. As one example, the matrix can be formed [symbol] as follows:

[symbol]

=qr(S′) (2);

where qr is the QR-factorization. For the ultrasound blood flow imaging applications where the tissue signal is the most dominant signal followed by blood signal and then noise, the approximate basis formed by [symbol] will satisfy the following relationship:

S≈ [symbol] [symbol] *S (3);

where [symbol] * is the complex conjugate transpose of the [symbol] matrix. Based on this representation of the ultrasound data based on the randomized data contained in the [symbol] matrix, the tissue clutter signal can be estimated, as indicated at step 310, which can then be used to estimate the clutter filtered blood flow signal, as indicated at step 312. From the estimated blood flow signal, images of blood flow in the subject can be produced. The estimated blood flow signal, or blood flow images produced therefrom, can further be processed. As one example, the estimated blood flow signal, or blood flow images produced therefrom, can be processed to compensate for non-uniform noise distributions, such as by using the methods described in co-pending International Patent Application Serial No. PCT/US2017/16190, which is herein incorporated by reference in its entirety.

For the purpose of ultrasound clutter filtering, it can generally be assumed that tissue clutter primarily resides in the first k rank of the singular values. Based on this assumption, the tissue clutter signal, T, can be represented by,

T= [symbol] [symbol] *S (4);

and the blood signal, B, can be obtained by,

B=S−T=S− [symbol] [symbol] *S (5).

As another example, the blood and tissue clutter signals can be obtained based on a singular value decomposition of the matrix [symbol] *S,

[symbol]

*S=ŨDV* (6);

where D contains the approximated first k singular values in the diagonal elements, V contains the right singular vectors, and the left singular vectors, U, can be obtained from U= [symbol] U. Using this approach, the tissue clutter signal can be obtained as,

T= [symbol] ŨDV* (7);

and the blood signal can be obtained as,

B=S− [symbol] ŨDV* (8).

The matrix [symbol] *S represents the projection of the ultrasound data onto the low-dimensional subspace defined by the randomized ultrasound data, S′. Because the matrix [symbol] *S has a dimension of (k+r)×n, which is typically much smaller than m×n, a full SVD on [symbol] *S is much faster than a full SVD on the original data matrix, S. Although the computational cost of the full SVD on the much smaller matrix [symbol] *S is significantly less than on the original data matrix, S, in practice if singular values and vectors are not needed or otherwise desired, Eqn. (5) can provide better computational performance because a full SVD does not need to be performed.

As one example instance where it may be desirable to compute singular values and singular vectors, the first k singular values and singular vectors can be used to determine an adaptive SVT cutoff value as described above. In those instances where the SVT cutoff value is greater than k, singular values with order of k+1, k+2, k+3, and so on, can be incrementally calculated using rSVD methods until a desired SVT cutoff value is reached. As another example, another k-orders of singular values can be calculated on top of the already calculated k singular values (i.e., reaching singular values at the order of 2k), and this process can be incrementally repeated until a desired SVT cutoff value is reached.

The advantage of using the rejected tissue signal as the background B-mode image is that no additional B-mode sequences need to be acquired to provide the background signal, which reduces the amount of time needed to obtain a blood flow image with B-mode background, which in turn improves the blood flow imaging frame rate. The same set of ultrasound data can be used to provide both the blood flow signal and the background B-mode signal (e.g., the tissue signal, T, as in Eqn. (7)) as the anatomical references. Another option for displaying the background B-mode signal is to use the original ultrasound data before clutter filtering. For example, IQ data can be used to obtain the B-mode image, and then the same IQ data can be used for clutter filtering to obtain a blood flow image.

To increase the singular value decay rate of [symbol] and improve the accuracy of the rSVD decomposition, a sequential power iteration can be used. For each power iteration, the following steps are executed,

[symbol]

ᵢ=qr(S* [symbol] ) (9);

[symbol]

=qr(S [symbol] ᵢ) (10).

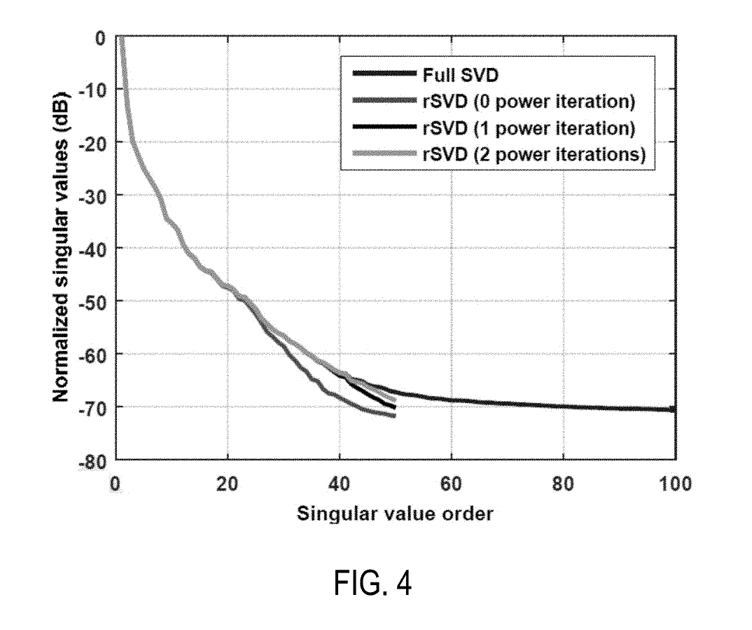

As shown in FIG. 4, a regular rSVD of a typical ultrasound microvessel imaging data without power iteration and with k=50 results in a discrepancy between the approximated singular value curve and the full SVD-estimated singular value curve. The first power iteration significantly decreases the difference between the rSVD curve and the full SVD curve. The second power iteration also improves the accuracy. Because each power iteration involves two QR factorizations, it is computationally expensive. In practice, it is contemplates that it is sufficient to perform only one power iteration to balance the tradeoff between accuracy and computational cost.

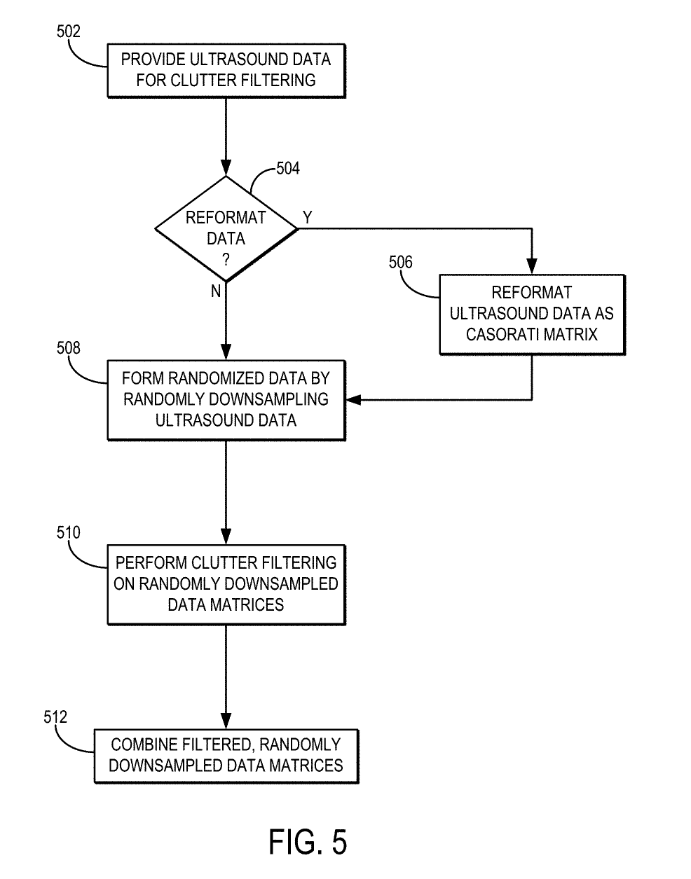

Referring now to FIG. 5, a flowchart is illustrated at setting forth the steps of an example method for accelerated clutter filtering of ultrasound data using a randomized spatial downsampling of the ultrasound data. The original ultrasound data matrix, S, which has dimension x×y×t (where X and y correspond to lateral and axial dimensions, respectively, and t corresponds to the temporal dimension) corresponding to the data acquired with the ultrasound system are provided for processing, as indicated at step 502. In some implementations, the ultrasound data matrix can be reformatted as a Casorati matrix with dimension xy×t before processing, as determined at decision block 504 and indicated at step 506. As described above, the dimensions of the Casorati matrix can also be defined as m×n with m=xy and n=t. In instances where the ultrasound data include three spatial dimensions (i.e., x, y, and z), the Casorati matrix will have dimensions m×n with m=xyz and n=t.

For singular value-based clutter filtering, dividing the large region-of-interest (“ROI”) into smaller non-overlapping or overlapping blocks will cause inconsistent clutter rejection among different blocks. As a result, the resulting blood flow image will be significantly deteriorated with patchy artifacts. To facilitate more robust clutter filtering with downsampled data, the singular value characteristics of the original data for each downsampled data set are preserved by implementing a randomized spatial downsampling of the ultrasound data, which promotes consistent clutter rejection across all downsampled data sets and prevents artifacts.

Randomized data are thus generated by randomly downsampling the ultrasound data, as indicated at step 508. In general, the randomized data includes multiple different randomly downsampled data sets. The downsampling of the ultrasound data can be executed on a parallel processing environment (e.g., a multi-thread or multi-core processor, a cluster, a graphics processing unit (“GPU”)) so that the randomly downsampled matrices can be parallel processed for accelerated computational performance.

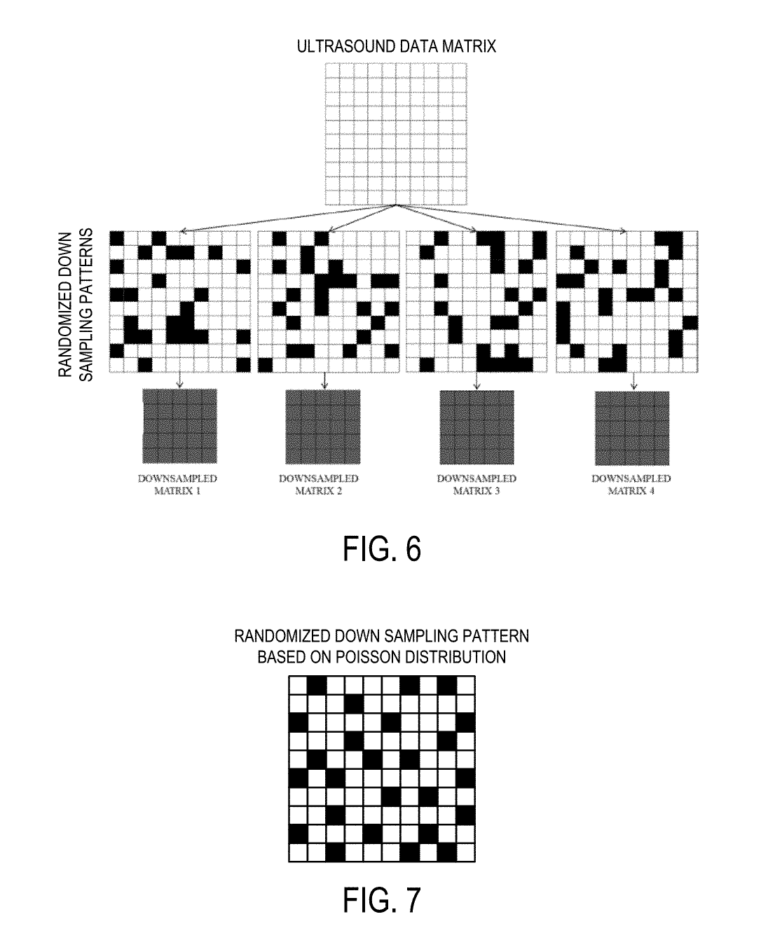

FIG. 6 illustrates one example method for randomly downsampling the ultrasound data, in which samples of the ultrasound data are randomly selected to form downsampled data matrices. This method can avoid stripe artifacts that can appear with structured downsampling methods. The randomized downsampling does not have any stripe artifacts even with very high downsample rate. The randomized downsampling can be performed in the Casorati matrix domain, as indicated above. The matrix elements within each randomly downsampled data matrix can be mutually exclusive or can be allowed to at least partially overlap. The downsampling process can be arranged to guarantee that each element of the original ultrasound data matrix is included at least once in a certain randomly downsampled matrix; however, this is not a requirement.

FIG. 7 illustrates another example method for randomly downsampling the ultrasound data, in which the randomized downsampling of the ultrasound data is based on a Poisson distribution that can provide more evenly distributed samples as compared to the method described above with respect to FIG. 6. For example, the random sampling pattern can be based on a Poisson Disk or similar distribution that targets a blue noise power spectrum. In this approach, the distance between each pair of samples is at least a distance, d, apart. The value of this distance, d, can be specified by a user. The Poisson randomized downsampling approach also provides the benefit of not producing stripe artifacts. Based on a desired downsampled rate, a number of randomly downsampled matrices following the Poisson random sampling distribution will be generated. The matrix elements within each randomly downsampled matrix can be mutually exclusive or can be allowed to at least partially overlap. The downsampling process can be arranged to guarantee that each element of the original ultrasound data matrix is included at least once in a certain randomly downsampled matrix; however, this is not a requirement.

Referring again to FIG. 5 clutter filtering is applied to the randomly downsampled data matrices, as indicated at step 510. As one example, the randomly downsampled data matrices can be processed according to the randomized SVD methods described herein with respect to FIG. 3. In these instances, the input data for the randomized SVD methods will be the randomly downsampled data matrices rather than the original ultrasound data. The randomly downsampled data matrices can also be processed using other clutter filtering methods.

The clutter filtered randomly downsampled matrices are then combined, as indicated at step 512, to produce an estimate of the blood flow signal. As an example, the combination of the randomly downsampled data matrices can include assigning the blood flow signals from each randomly downsampled matrix to the correct location of the final blood signal matrix based on the location of each element in the original ultrasound data matrix. If a certain element of the original ultrasound data matrix is included in more than one of the randomly downsampled data matrices, an average value of the blood flow signal among the randomly downsampled matrices that include the element will be assigned to the correct location of the final blood flow signal matrix. From the estimated blood flow signal, images of blood flow in the subject can be produced. The estimated blood flow signal, or blood flow images produced therefrom, can further be processed. As one example, the estimated blood flow signal, or blood flow images produced therefrom, can be processed to compensate for non-uniform noise distributions, such as by using the methods described in co-pending International Patent Application Serial No. PCT/US2017/16190, which is herein incorporated by reference in its entirety.

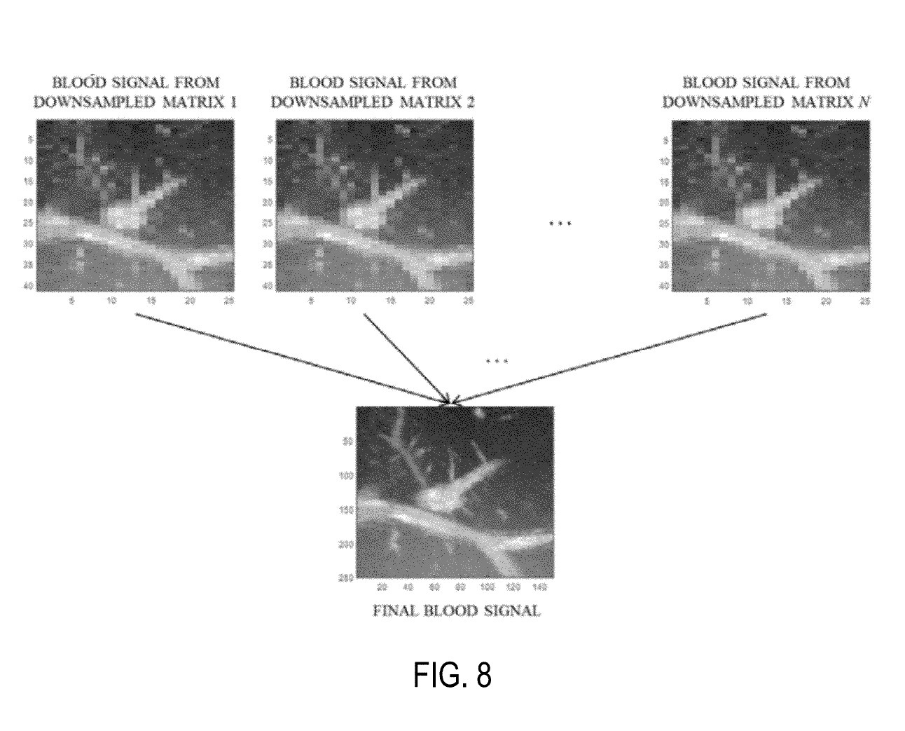

An example of combining blood flow signals from downsampled matrices into a final blood signal is shown in FIG. 8. Because of the much smaller size of each downsampled matrix, the computational cost of clutter filtering is much less for each downsampled matrix. It is contemplated that an acceleration factor of N (i.e., the number of downsampled matrices) can be achieved with parallel processing.

Although methods for randomized spatial downsampling are described above, it will be appreciated by those skilled in the art that structured downsampling of the ultrasound data can also provide benefits for the randomized SVD clutter filtering described above with respect to FIG. 3. In these instances, the ultrasound data can be downsampled using a structured downsampling pattern and the resulting downsampled data matrices can be processed according to the randomized SVD methods described above (i.e., the input data for the randomized SVD methods will be the downsampled data matrices rather than the original ultrasound data).

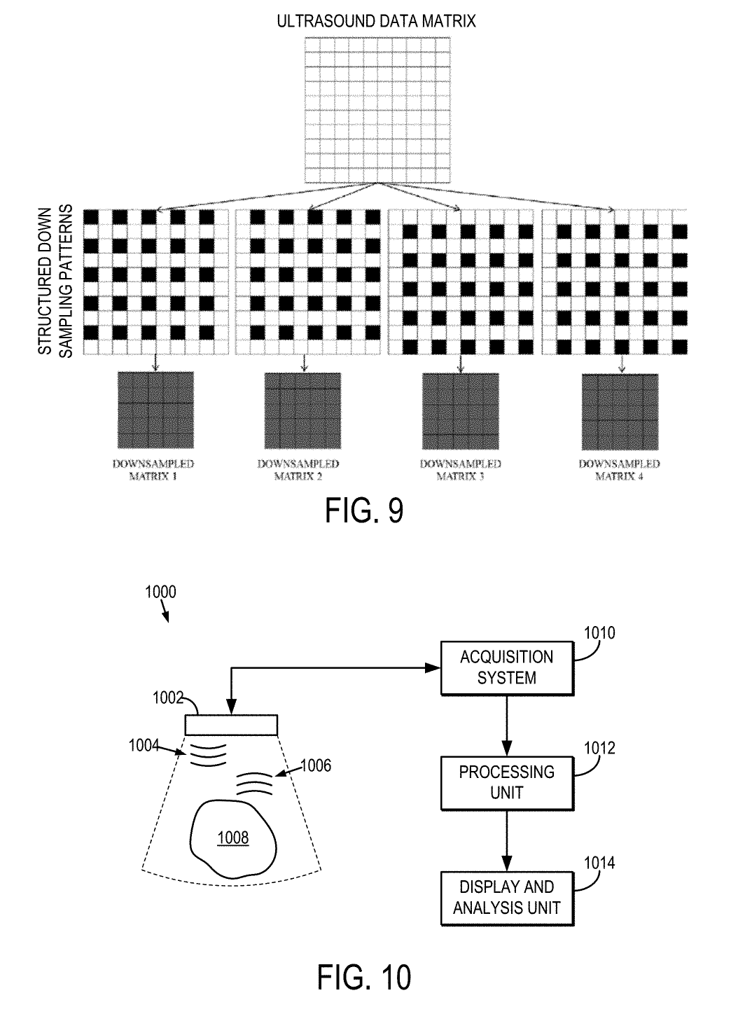

Downsampling the ultrasound data generates multiple different downsampled matrices each having a smaller matrix size than the original ultrasound data, which can accelerate clutter filtering processing. As one example, downsampling the ultrasound data can include selecting every other sample along the row and along the column for each downsampled matrix, as depicted in FIG. 9. The downsampling factor in each dimension in this example is 2, with a combined downsampling factor of 4. The downsampling factor along each dimension can be any arbitrary positive integer number that is smaller than or equal to the dimension size of the matrix. The matrix elements within each downsampled matrix can be mutually exclusive or properly overlapped for a smooth visual appearance of the final combined blood flow image.

Because of data redundancy, each downsampled matrix has similar singular value characteristics to the original data matrix, and therefore a robust singular value-based clutter filtering can be performed on each downsampled matrix. The resulting blood flow signal matrices can then be combined by reversing the downsample process to form the final blood flow signal matrix.

FIG. 10 illustrates the main components of an example ultrasound imaging system 1000 that can implement the methods described here. The system 1000 generally includes an ultrasound transducer 1002 that transmits ultrasonic waves 1004 and receives ultrasonic echoes 1006 from an object 1008, which may be tissue in a subject. An acquisition system 1010 acquires ultrasound signals from the transducer 1002 and outputs the signals to a processing unit 1012, which can include a suitable computer system or processor. In some implementations, the acquisition system 1010 beamforms the signal from each transducer element channel and outputs the signal to the processing unit 1012. The processing unit 1012 can be programmed to implement the methods described here for generating images that depict or quantify blood flow in a subject's vasculature, including in small vessels. The output from the processing unit 1012 can be displayed and analyzed by a display and analysis unit 1014, which can include a suitable computer display or computer system.

The acquisition system 1010 can have a high imaging frame and volume rate, such that the acquisition pulse-repetition-frequency (“PRF”) can be at least 100 Hz. The system 1000 can sample and store at least one hundred ensembles of ultrasound signals in the temporal direction. The ultrasound system 1000 can transmit and receive at least one of focused waves, diverged waves, spherical waves, cylindrical waves, and plane waves. The ultrasound system 1000 can implement a detection sequence that includes one of conventional line-by-line scanning, compounding plane wave imaging, compounding diverging beam imaging, and synthetic transmit aperture imaging. Furthermore, the transmit pulses generated by the ultrasound system 1000 can include at least one of conventional non-coded imaging pulses and spatially or temporally encoded pulses. The receive pulses generated by the ultrasound system 1000 can in some instances be generated based on at least one of fundamental frequency and harmonic frequencies.

Although the above teachings are given in the contexts of ultrasound blood flow imaging, the methods disclosed here can be used to accelerate any applications that use singular value decomposition, such as in B-mode ultrasound imaging or in other imaging modalities and signal processing applications.

The present disclosure has described one or more preferred embodiments, and it should be appreciated that many equivalents, alternatives, variations, and modifications, aside from those expressly stated, are possible and within the scope of the invention.

1. A method for estimating a blood flow signal from ultrasound data acquired using an ultrasound imaging system, the steps of the method comprising:
(a) providing ultrasound data acquired from a subject with the ultrasound imaging system;
(b) forming randomized data by randomizing the ultrasound data; and
(c) estimating blood flow signal data from the ultrasound data by clutter filtering tissue signals from the ultrasound data using the randomized data.

2. The method as recited in claim 1, wherein forming the randomized data includes multiplying the ultrasound data by a random matrix.

3. The method as recited in claim 2, wherein the random matrix has at least one dimension that is a sum of a first rank associated with a subspace in which tissue clutter signals are expected to reside and an additional rank.

4. The method as recited in claim 3, wherein the additional rank is one of 1 or 2.

5. The method as recited in claim 2, wherein step (c) includes calculating a Q-matrix whose columns form an orthonormal basis for a column space of the ultrasound data using the randomized data, and filtering the tissue signals from the ultrasound data using the calculated matrix.

6. The method as recited in claim 5, wherein the Q-matrix is calculated from the randomized data using a QR factorization.

7. The method as recited in claim 5, wherein the Q-matrix is calculated from the randomized data using a power iteration.

8. The method as recited in claim 5, wherein filtering the tissue signals from the ultrasound data using the Q-matrix includes:
multiplying the ultrasound data by a complex conjugate of the Q-matrix to form a second matrix;
multiplying the second matrix by the Q-matrix to estimate tissue signal data; and
subtracting the tissue signal data from the ultrasound signal data to estimate the blood flow signal data.

9. The method as recited in claim 5, wherein filtering the tissue signals from the ultrasound data using the Q-matrix includes:
multiplying the ultrasound data by a complex conjugate of the Q-matrix to form a second matrix;
computing a singular value decomposition of the second matrix;
multiplying the singular value decomposition of the second matrix to estimate tissue signal data; and
subtracting the tissue signal data from the ultrasound signal data to estimate the blood flow signal data.

10. The method as recited in claim 1, wherein the randomized data comprises a plurality of randomized data sets and step (b) includes downsampling the ultrasound data using a number of different downsampling patterns to generate a plurality of downsampled data sets and forming the plurality of randomized data sets by multiplying each downsampled data set by a random matrix.

11. The method as recited in claim 10, wherein each of the number of different downsampling patterns represents a structured downsampling pattern.

12. The method as recited in claim 10, wherein each of the number of different downsampling patterns represents a random downsampling pattern.

13. The method as recited in claim 10, wherein at least some of the different downsampling patterns include sampling points that spatially overlap.

14. The method as recited in claim 10, wherein the different downsampling patterns collectively include sampling points that sample mutually exclusive spatial locations.

15. The method as recited in claim 10, wherein the random matrix has at least one dimension that is a sum of a first rank associated with a subspace in which tissue clutter signals are expected to reside and an additional rank.

16. The method as recited in claim 10, wherein step (c) includes for each randomized data set calculating a Q-matrix whose columns form an orthonormal basis for a column space of the downsampled data set associated with the randomized data set, and wherein filtering the tissue signals from the ultrasound data includes using each Q-matrix.

17. The method as recited in claim 16, wherein filtering the tissue signals from the ultrasound data using each Q-matrix includes:
multiplying each downsampled data set by a complex conjugate of the Q-matrix associated with the downsampled data set to form a second matrix;
multiplying each second matrix by the Q-matrix associated with the second matrix to estimate tissue signal data;
combining the tissue signal data associated with each downsampled data set; and
subtracting the tissue signal data from the ultrasound signal data to estimate the blood flow signal data.

18. The method as recited in claim 16, wherein filtering the tissue signals from the ultrasound data using the Q-matrix includes:
multiplying each downsampled data set by a complex conjugate of the Q-matrix associated with the downsampled data set to form a second matrix;
computing a singular value decomposition of each second matrix;
multiplying each singular value decomposition of each second matrix by the Q-matrix associated with the second matrix to estimate tissue signal data;
combining the tissue signal data associated with each downsampled data set; and
subtracting the tissue signal data from the ultrasound signal data to estimate the blood flow signal data.

19. The method as recited in claim 1, wherein the randomized data comprises a plurality of randomly downsampled data sets.

20. The method as recited in claim 19, wherein each of the plurality of randomly downsampled data sets is formed by downsampling the ultrasound data using a different random downsampling pattern.

21. The method as recited in claim 20, wherein each random downsampling pattern is based on a distribution that targets a blue noise power spectrum.

22. The method as recited in claim 21, wherein the random downsampling pattern is based on a Poisson Disk.

23. The method as recited in claim 1, wherein step (b) includes converting the ultrasound data to a Casorati matrix and step (b) includes forming the randomized data from the Casorati matrix.

24. The method as recited in claim 1, further comprising producing an image of blood flow in the subject from the estimated blood flow signal data.
